(19) United States
(12) Patent Application Publication
(10) Pub. No.: US 2017/0367865 A1
(43) Pub. Date: Dec. 28, 2017

(54) Ankle reinforcing device

(71) Applicant: Richard Michael Truhill, Plano, TX (US)

(72) Inventor: Richard Michael Truhill, Plano, TX (US)

(21) Appl. No.: 14/999,755

(22) Filed: Jun. 24, 2016

(51) Int. Cl. A61F 5/01; A63B 71/12

(52) U.S. Cl. CPC A61F 5/0111; A63B 71/1225; A61F 2005/0181; A63B 2071/1275

(57) ABSTRACT

My invention is an ankle reinforcing device for the prevention of medial and/or lateral ankle injuries such as sprains or breaks. The object of the invention is to provide an inexpensive but highly effective device which will allow free and unhampered movement of the wearer's foot while engaged in athletic events or other physical activity but will prevent movement in such a direction and to such an extent that would result in spraining or breaking an ankle. The ankle reinforcing device is constructed from an underlying member which covers all or part of the foot and part of the leg above the ankle, reinforcing members attached to or integral with the underlying member, and means to securely attach the device to the wearer. The reinforcing members are arranged in a pattern to prevent excessive movement of the ankle in the medial and/or lateral directions, while allowing free up and down movement of the foot.

### BACKGROUND OF THE INVENTION

### 1. Field of the Invention

The present invention relates to an ankle reinforcing device for the prevention of ankle injuries such as sprains or breaks.

Ankle injuries are not uncommon among those engaged in physical activities such as sports or dancing, or even just wearing high-heels. Many devices have been made available to help prevent ankle injuries. However, such devices have several drawbacks: they tend to be bulky and overly restrict movement of the foot.

Various ankle injuries can occur, but most involve lateral (outside) or medial (inside) sprains. Some athletes, such as professionals, college athletes, and some high-school athletes, have access to athletic trainers or others who can “tape” the athletes' ankles; however, most people do not have that option. Reusable ankle support devices tend to be expensive and impractical for many athletes and dancers because of the drawbacks previously mentioned.

Some devices use adhesives directly on the wearer's skin, but most users prefer to avoid this practice for various reasons such as skin sensitivity and difficulty removing. Taping of ankles is usually done in such a way that adhesives do not come in direct contact with the skin. The proposed invention does not require any adhesive to come in contact with the skin. The disposable model is easily removed simply by cutting the fastening tape. The reusable model is easily removed simply by un-wrapping the fastening tape.

### 2. Description of Prior Art

Various types of ankle supports and braces are known in the prior act to provide means to prevent ankle injuries.

U.S. Pat. No. 3,357,425 issued to Morgan Dec. 12, 1967 discloses an adhesive support wrap to prevent ankle injuries adhered directly to the wearer's skin.

U.S. Pat. No. 4,345,590 issued to Nakajima Aug. 24, 1982 discloses a pre-cut adhesive support bandage which attaches directly to the leg and foot.

U.S. Pat. No. 4,522,044 issued to Lineberry, et al. Jun. 11, 1985 discloses a sock with ankle support woven into the sock.

U.S. Pat. No. 4,753,229 issued to Sutherland Jun. 28, 1988 discloses a reusable ankle brace with reinforcing straps attached to a shoe.

U.S. Pat. No. 5,217,431 issued to Toronto, et al. Jun. 8, 1993 discloses an ankle brace consisting of a boot.

U.S. Pat. No. 5,617,745 issued to Della Corte, et al. Apr. 8, 1997 discloses a support sock with an ankle stabilization section woven in.

U.S. Pat. No. 5,944,678 issued to Hubbard, et al. Aug. 31, 1999 discloses an ankle brace with elastic connections between the foot section and the upper portion.

It is apparent from the foregoing that the prior art fails to teach an ankle reinforcing device as described herein: possessing an underlying member having flexible areas which allow the wearer's foot to move up and down at the ankle during use; reinforcing members consisting of high tensile strength materials such as metal, fiberglass, carbon fiber, aramid fiber, or other high tensile strength material, which are integral with or attached to said underlying member and arranged in a radial pattern to simultaneously restrict movement of the ankle in the medial and/or lateral directions while forming a hinge at the ankle such that up and down movement of the foot at the ankle is not greatly restricted; means to optimally position said ankle reinforcing device; means to securely fasten said ankle reinforcing device to the foot and the leg; markings to facilitate the correct placement of said means to securely fasten said ankle reinforcing device to the foot and the leg; and means to increase friction between said ankle reinforcing device and the wearer's skin. Said ankle reinforcing device being either disposable (for one-time use) or reusable (for repeated use); some or all of the materials used therein having a high coefficient of friction on human skin, such as latex, nitrile or silicone; some or all of the materials used therein being hypoallergenic; some or all of the materials used therein being waterproof; said ankle reinforcing device being small enough to be worn inside a sock or shoe; and said ankle reinforcing device being able to be worn on a range of foot sizes.

### SUMMARY OF THE INVENTION

An ankle reinforcing device is disclosed, comprising an underlying member; reinforcing members arranged to restrict movement of the ankle in the medial and/or lateral directions while allowing up and down movement of the foot at the ankle; means to securely fasten said ankle reinforcing device to the wearer's foot and the leg; means to position said ankle reinforcing device for optimal effectiveness; and means to increase friction between said ankle reinforcing device and the wearer's skin. Both disposable and reusable models are offered.

In practical use, the invention is placed on the wearer's foot in the same manner as putting on a sock, with positioning markings placed over the ankle joint. One or more fasteners are attached above the ankle joint. One or more fasteners are attached across the front of the foot, the back of the foot, and/or the bottom of the foot. Integral reinforcing members then limit movement of the wearer's ankle in the medial and/or lateral directions. Said reinforcing members are positioned to cross at the ankle, creating a hinge point so up and down movement of the foot at the ankle is allowed. The wearer is thus able to run, jump, and otherwise participate in physical activities according to his or her ability while reducing the likelihood of having an ankle injury.

One embodiment of the invention has reinforcing members on both the outside and the inside of the ankle, preventing lateral and medial ankle injuries. A second embodiment of the invention has reinforcing members only on the outside of the ankle, preventing lateral ankle injuries. Another embodiment of the invention has reinforcing members only on the inside of the ankle, preventing medial ankle injuries.

One embodiment of the invention uses adhesive tape to securely attach said ankle reinforcing device to the wearer's leg above the ankle and to the wearer's foot below the ankle. Another embodiment uses a self-adhering or cohesive bandage type wrap to secure said ankle reinforcing device to the wearer's foot and leg so it can be worn more than once. Another embodiment uses hook and pile type fasteners to secure said ankle reinforcing device to the wearer's foot and leg so it can be worn more than once.

In one embodiment, all or part of the materials that come in contact with the wearer's skin have a high coefficient of friction on human skin, such as latex, nitrile or silicone. For example, in one embodiment the entire underlying member is made of such material. In another embodiment said underlying member may be made of a natural material such as cotton, wool, etc., or synthetic fiber, allowing moisture to escape from the wearer's foot; and friction panels integral with or attached to the inside of said underlying member and/or integral with or attached to said reinforcing members may directly contact the wearer's skin. These embodiments serve to increase the effectiveness of the device in preventing ankle injuries.

The proposed invention can be installed very quickly by the user or by others, such as trainers or coaches. One embodiment incorporates markings to guide the installer in correctly positioning said means to securely fasten said ankle reinforcing device to the wearer's leg and foot.

Other aspects of the construction, arrangement and combination of the various parts of the invention whereby the desired objectives are obtained, as well as advantages of the invention, will appear from the following description in which the preferred embodiments are discussed in detail as illustrated by the accompanying drawings.

### BRIEF DESCRIPTION OF THE DRAWINGS

FIG. 1 illustrates one form of a disposable ankle reinforcing device, wherein said means to securely fasten said ankle reinforcing device comprise adhesive type tape.

FIG. 2 illustrates one form of a reusable ankle reinforcing device, wherein said means to securely fasten said ankle reinforcing device comprise self-adhering or cohesive bandage type wrap or hook and pile type fasteners.

FIG. 3 illustrates a disposable ankle reinforcing device as it is worn on the foot.

FIG. 4 illustrates a reusable ankle reinforcing device as it is worn on the foot and demonstrates a preferred tape wrapping pattern.

FIG. 5 illustrates friction panels which interact with the wearer's skin to prevent movement of the proposed device during use.

### DETAILED DESCRIPTION OF THE INVENTION

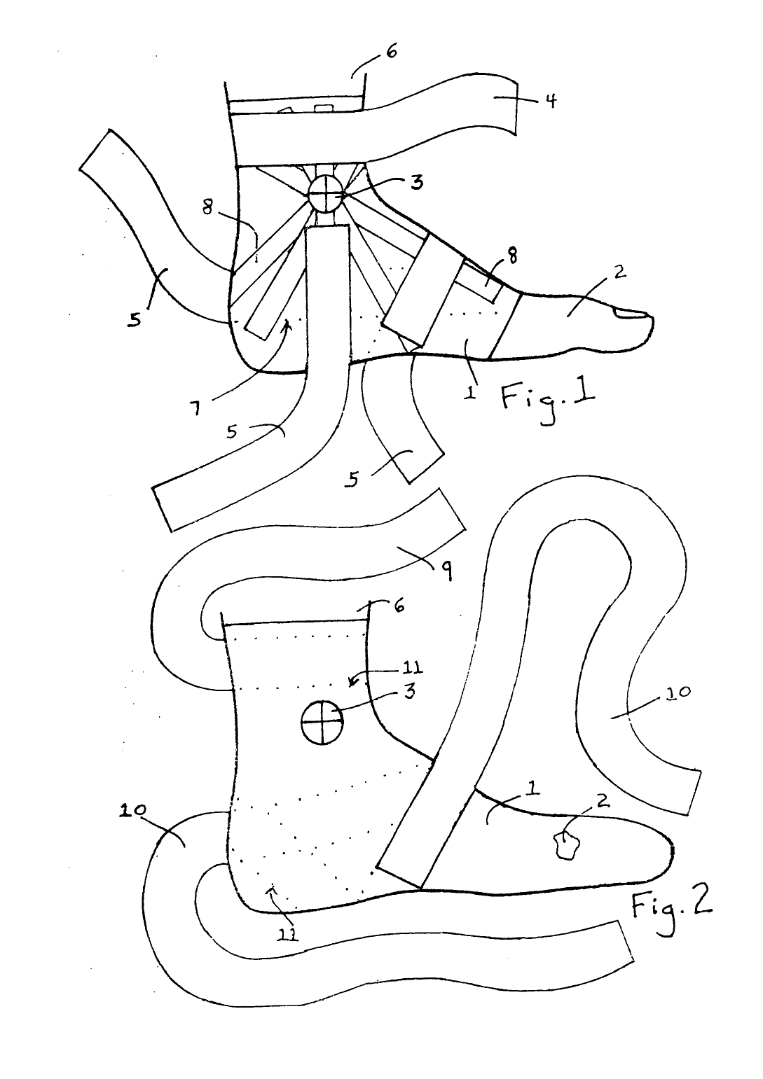

In the illustration of FIG. 1, an ankle reinforcing device is shown, as viewed from the medial side or inside of the leg, and includes an underlying member 1, which is worn on the foot 2. In this embodiment, said underlying member 1 only partially covers said foot 2. Said underlying member 1 is flexible so that it can be easily adjusted so positioning markings 3 can be placed over the ankle. Adhesive tape 4 securely attaches said ankle reinforcing device to the wearer's leg 6 above the ankle. Adhesive tapes 5 securely attach said ankle reinforcing device to the wearer's foot 2 below the ankle. Markings 7 facilitate the correct placement of said means 4 and 5 to securely fasten said ankle reinforcing device to the wearer's foot 2 and leg 6. Reinforcing members 8 are integral with and/or attached to said underlying member 1 and arranged to restrict movement of the ankle in the medial and/or lateral directions while allowing up and down movement of the foot at the ankle. Said adhesive tapes 4 and 5 are positioned to secure said reinforcing members 8, leg 6, and foot 2 so that movement of the ankle in the medial and/or lateral directions is restricted while allowing up and down movement of the foot at the ankle.

In the illustration of FIG. 2, an ankle reinforcing device is shown, as viewed from the medial side or inside of the leg, and includes an underlying member 1, which is worn on the foot 2. In this embodiment, said underlying member 1 completely covers said foot 2. Said underlying member 1 is flexible so that it can be easily adjusted so positioning markings 3 can be placed over the ankle. A removable self-adhering or cohesive bandage type wrap or a hook and pile type fastener 9 is used to securely attach said ankle reinforcing device to the wearer's leg 6 above the ankle. Additional removable self-adhering or cohesive bandage type wrap or hook and pile type fasteners 10 are used to securely attach said ankle reinforcing device to the wearer's foot 2 below the ankle. Markings 11 facilitate the correct placement of said means 9 and 10 to securely fasten said ankle reinforcing device to the wearer's foot 2 and leg 6. Reinforcing members (not shown) are integral with and/or attached to said underlying member 1 and arranged to restrict movement of the ankle in the medial and/or lateral directions while allowing up and down movement of the foot at the ankle. Said fasteners 9 and 10 are positioned to secure said reinforcing members (not shown), leg 6, and foot 2 so that movement of the ankle in the medial and/or lateral directions is restricted while allowing up and down movement of the foot at the ankle. Said fasteners 9 and 10 can be removed and re-applied, allowing said ankle reinforcing device to be used more than once, or its position to be adjusted during use.

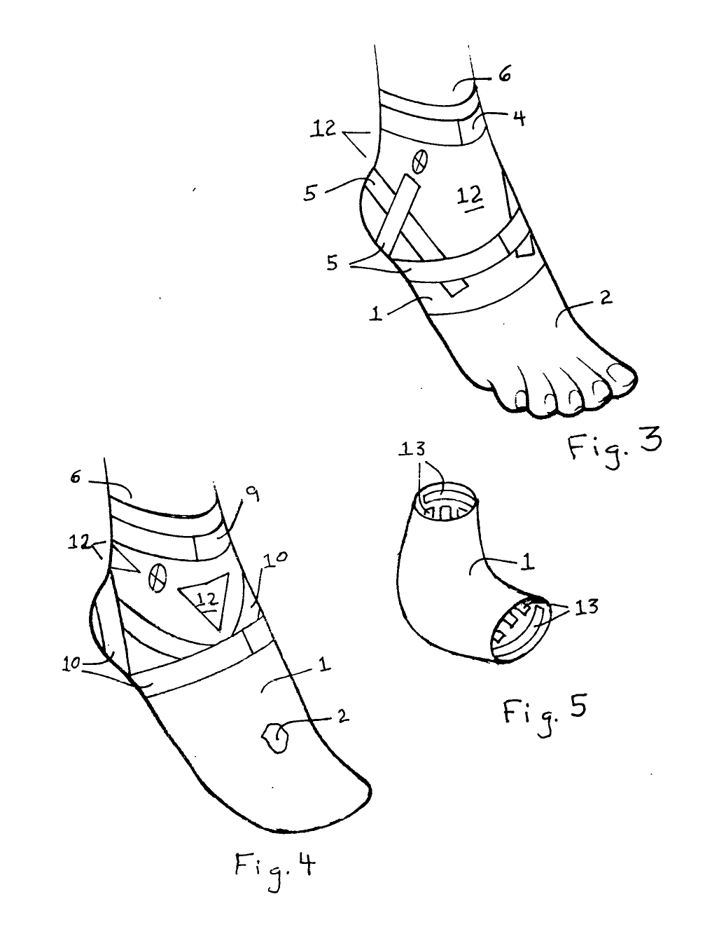

In the illustration of FIG. 3, an ankle reinforcing device is shown as it would appear when secured to the leg 6 and foot 2 of the wearer as viewed from the lateral side or outside of the leg. In this embodiment, said underlying member 1 only partially covers said foot 2. Adhesive tapes 4 and 5 are positioned to secure reinforcing members (not shown) to said leg 6 and foot 2 so that movement of the ankle in the medial and/or lateral directions is restricted while allowing up and down movement of the foot at the ankle. Adhesive tapes 4 and 5 can be provided as separate loose items, or they can be partially and permanently attached to said underlying member 1 so that the invention can be more easily installed on the wearer. A removable non-stick backing (not shown) protects adhesive areas of said adhesive tapes 4 and 5 until ready for use. Said non-stick backing (not shown) would be removed during installation of said ankle reinforcing device on the wearer's foot in order to attach said adhesive tapes 4 and 5. Areas 12 are flexible areas and/or open areas (not shown) of said underlying member 1, which are not restricted by said adhesive tapes 4 and 5 or by said reinforcing members (not shown), allowing the foot to move freely up and down at the ankle.

In the illustration of FIG. 4, a reusable ankle reinforcing device is shown as it would appear when secured to the leg 6 and foot 2 of the wearer as viewed from the lateral side or outside of the leg. In this embodiment, said underlying member 1 completely covers said foot 2. Self-adhering or cohesive bandage type wrap or hook and pile type fasteners 9 and 10 are positioned to secure reinforcing members (not shown), said leg 6, and said foot 2 so that movement of the ankle in the medial and/or lateral directions is restricted while allowing up and down movement of the foot at the ankle. Said fasteners 9 and 10 can be provided as separate loose items, or they can be partially and permanently attached to said underlying member 1 so that the invention can be more easily installed on the wearer. Areas 12 are flexible areas (not shown) and/or open areas of said underlying member 1, which are not restricted by said fasteners 9 and 10 or by said reinforcing members (not shown), allowing said foot 2 to move freely up and down at the ankle. A preferred wrapping pattern is shown for said fasteners 9 and 10, whereby said reinforcing members (not shown), leg 6, and foot 2 are secured such that movement of the ankle in the medial and/or lateral directions is restricted while allowing up and down movement of the foot at the ankle.

In the illustration of FIG. 5, the underlying member 1 of an ankle reinforcing device is shown to include friction panels 13 which are integral with or attached to the inside of said underlying member 1 and/or integral with or attached to reinforcing members (not shown). In this embodiment said underlying member 1 may be made of a natural material such as cotton, wool, etc. or synthetic material, allowing moisture to escape from the wearer's foot.

The various embodiments of the present invention provide for medial and/or lateral support of the wearer's ankle to prevent ankle injuries. The invention is adaptable to any size foot. The individual wearer may decide on the degree of tightness with which the ankle reinforcing device is attached. On the reusable model, re-adjustment is very simple. Use of the invention does not require the assistance of additional personnel.

The ankle reinforcing device of the present invention is very effective in accomplishing its purpose; yet it can be produced inexpensively. Due to its flexibility, the invention can be manufactured to fit a range of foot sizes, and various models can each cover different ranges of sizes. The design and the materials are such that the invention can easily be worn inside of any sock and/or shoe without discomfort.

Other alternative embodiments which achieve equivalent features of the present invention would be apparent to those skilled in the art. From the above description, it is intended that this disclosure be taken in an exemplary sense, and the scope of protection afforded be determined by the appended claims.

What is claimed is:

1. A disposable ankle reinforcing device, which comprises in combination:
an underlying member which is flexible and covers all or part of the wearer's foot and part of the wearer's leg;
reinforcing members integral with or attached to said underlying member and arranged in a radial pattern to restrict movement of the ankle either in the medial direction or in the lateral direction or in both directions to prevent over-extension of the ankle which could cause injury to the ankle, while up and down movement of the foot at the ankle is not greatly restricted;
means to position said ankle reinforcing device during installation on the wearer's foot so that said reinforcing members may optimally restrict movement of the ankle to prevent injury;
means to securely fasten said ankle reinforcing device to the foot and the leg comprising tape with a strong adhesive, allowing for one-time use, such that said tape with a strong adhesive engages said reinforcing members, either directly or in conjunction with said underlying member, and secures said reinforcing members to said wearer's foot and leg resulting in restricted movement of the ankle in the medial direction or in the lateral direction or in both directions, while allowing up and down movement of the foot at the ankle;
markings to facilitate the correct positioning of said means to securely fasten said ankle reinforcing device to the foot and the leg; and
means to increase friction between said ankle reinforcing device and the wearer's skin.

2. The ankle reinforcing device of claim 1, wherein said tape with a strong adhesive has a non-stick backing which is removed during installation of the invention in order to attach said tape.

3. The ankle reinforcing device of claim 1, wherein after installation on the wearer's foot said ankle reinforcing device contains flexible areas or open areas which allow the wearer's foot to move up and down at the ankle.

4. The ankle reinforcing device of claim 1, wherein said reinforcing members comprise high tensile strength materials such as metal, fiberglass, carbon fiber, aramid fiber, or other high tensile strength materials.

5. The ankle reinforcing device of claim 1, wherein said means to increase friction between said ankle reinforcing device and the wearer's skin have a high coefficient of friction on human skin, such as latex, nitrile or silicone, or other high coefficient of friction materials; and said materials comprise all or part of said ankle reinforcing device, including but not limited to underlying member materials, reinforcing member materials, etc.

6. The ankle reinforcing device of claim 1, wherein said means to increase friction between said ankle reinforcing device and the wearer's skin comprise panels integral with or attached to said underlying member or said reinforcing members; and said panels have a high coefficient of friction on human skin.

7. The ankle reinforcing device of claim 1, wherein all or part of said ankle reinforcing device (including but not limited to underlying member materials, reinforcing member materials, means to securely fasten materials, etc.) are hypoallergenic.

8. The ankle reinforcing device of claim 1, wherein all or part of said ankle reinforcing device (including but not limited to underlying member materials, reinforcing member materials, means to securely fasten materials, etc.) are waterproof.

9. The ankle reinforcing device of claim 1 which is of such compact size that it can be worn inside of common footwear such as socks and shoes.

10. The ankle reinforcing device of claim 1 which fits a range of foot sizes.

11. A reusable ankle reinforcing device, which comprises in combination:
an underlying member which is flexible and covers all or part of the wearer's foot and part of the wearer's leg;
reinforcing members integral with or attached to said underlying member and arranged in a radial pattern to restrict movement of the ankle either in the medial direction or in the lateral direction or in both directions to prevent over-extension of the ankle which could cause injury to the ankle, while up and down movement of the foot at the ankle is not greatly restricted;
means to position said ankle reinforcing device during installation on the wearer's foot so that said reinforcing members may optimally restrict movement of the ankle to prevent injury;
means to securely fasten said ankle reinforcing device to the foot and the leg comprising self-adhering or cohesive bandage type wrap or hook and pile type fasteners, allowing said ankle reinforcing device to be used more than once, such that said self-adhering or cohesive bandage type wrap or hook and pile type fasteners engage said reinforcing members, either directly or in conjunction with said underlying member, and secure said reinforcing members to said wearer's foot and leg resulting in restricted movement of the ankle in the medial direction or in the lateral direction or in both directions, while allowing up and down movement of the foot at the ankle;
markings to facilitate the correct positioning of said means to securely fasten said ankle reinforcing device to the foot and the leg; and
means to increase friction between said ankle reinforcing device and the wearer's skin.

12. The ankle reinforcing device of claim 11, wherein after installation on the wearer's foot said ankle reinforcing device contains flexible areas or open areas which allow the wearer's foot to move up and down at the ankle.

13. The ankle reinforcing device of claim 11, wherein said reinforcing members comprise high tensile strength materials such as metal, fiberglass, carbon fiber, aramid fiber, or other high tensile strength materials.

14. The ankle reinforcing device of claim 11, wherein said means to increase friction between said ankle reinforcing device and the wearer's skin have a high coefficient of friction on human skin, such as latex, nitrile or silicone, and said means to increase friction comprise all or part of said ankle reinforcing device, including but not limited to underlying member materials, reinforcing member materials, etc.

15. The ankle reinforcing device of claim 11, wherein means to increase friction between said ankle reinforcing device and the wearer's skin comprise panels integral with or attached to said underlying member or said reinforcing members; and said panels have a high coefficient of friction on human skin.

16. The ankle reinforcing device of claim 11, wherein all or part of said ankle reinforcing device (including but not limited to underlying member materials, reinforcing member materials, means to securely fasten materials, etc.) are hypoallergenic.

17. The ankle reinforcing device of claim 11, wherein all or part of said ankle reinforcing device (including but not limited to underlying member materials, reinforcing member materials, means to securely fasten materials, etc.) are waterproof.

18. The ankle reinforcing device of claim 11 which is of such compact size that it can be worn inside of common footwear such as socks and shoes.

19. The ankle reinforcing device of claim 11 which fits a range of foot sizes.

20. A method to reinforce human ankles and protect ankles from injury comprising:
an underlying member which is flexible and covers all or part of the wearer's foot and part of the wearer's leg;
reinforcing members integral with or attached to said underlying member and arranged in a radial pattern to restrict movement of the ankle either in the medial direction or in the lateral direction or in both directions to prevent over-extension of the ankle which could cause injury to the ankle, while up and down movement of the foot at the ankle is not greatly restricted;
means to position said ankle reinforcing device during installation on the wearer's foot so that said reinforcing members may optimally restrict movement of the ankle to prevent injury;
means to securely fasten said ankle reinforcing device to the foot and the leg which engages said reinforcing members, either directly or in conjunction with said underlying member, to secure said reinforcing members to said wearer's foot and leg resulting in restricted movement of the ankle in the medial direction or in the lateral direction or in both directions, while allowing up and down movement of the foot at the ankle;
markings to facilitate the correct positioning of said means to securely fasten said ankle reinforcing device to the foot and the leg; and
means to increase friction between said ankle reinforcing device and the wearer's skin.
